SEQUENCE LISTING (4 sequences)

SEQ ID NO: 1 - DNA, 33 bp, Artificial
  Synthesized
ggaattccatatggtacgcgatggttatattgc

SEQ ID NO: 2 - DNA, 29 bp, Artificial
  Synthesized
ccgctcgagaccgccattgcattttcctg

SEQ ID NO: 3 - DNA, 51 bp, Artificial
  Synthesized
ggaattccatatgcatcatcatcatcatcacgtacgcgatggttatattgc

SEQ ID NO: 4 - DNA, 32 bp, Artificial
  Synthesized
cgcggatccttaaccgccattgcattttcctg

(19) United States
(12) Patent Application Publication
(10) Pub. No.: US 2017/0002048 A1
(43) Pub. Date: Jan. 5, 2017

(54) METHOD FOR PURIFYING AND RENATURATING INCLUSION BODIES OF SCORPION TOXIN PROTEIN AND THEIR USE

(71) Applicant: GUANGZHOU GLAM BIOTECHNOLOGY CO., LTD., Guangzhou City (CN)

(72) Inventor: Li Li, Guangzhou City (CN)

(21) Appl. No.: 15/158,771

(22) Filed: May 19, 2016

(30) Foreign Application Priority Data
Jul. 2, 2015 (CN) 201510386346.4

(51) Int. Cl. C07K 14/435; C07K 1/22; C07K 1/113

(52) U.S. Cl. CPC C07K 14/43522; C07K 1/1136; C07K 1/22; A61K 38/00

(57) ABSTRACT

A method for purifying and renaturating inclusion bodies of scorpion venom protein is provided. The method includes expressing the scorpion venom protein by recombinant Escherichia coli. The C-terminal of the scorpion venom protein has His-tag. The method includes breaking the disulfide bonds in the scorpion venom protein by a denaturating buffer, purifying the denaturated scorpion venom protein with a histidine affinity chromatography column, and renaturating the scorpion venom protein with a renaturation buffer. The renaturation buffer has a pH of 7-9 and includes 50-200 mmol/L Na₂HPO₄, 10-100 mmol/L Tris, 0.1-1 mol/L L-Arg, 1-5 mmol/L EDTA, 0.1-5 mmol/L GSH, 0.05-0.5 mmol/L GSSG, 5-20% (v/v) glycerol, 0.01-5% (v/v) triton X-100. Preparation of the scorpion venom protein by this method has the advantages of simple operation, and good renaturation effect.

### TECHNICAL OF THE INVENTION

The present invention belongs to the technical field of bioengineering, specifically it relates to a method for purifying and renaturating inclusion bodies of scorpion toxin protein and their use.

### BACKGROUND OF THE INVENTION

In China, scorpion is a traditional Chinese medicine, it has a very high medicinal value. In Compendium of Materia Medica it is recorded to treat “all wind with vertigo and shaking, hyperspasmia, malarial heat and cold, and deaf without hearing”; in Illustrated Classics of Materia Medica it is recorded to treat “paediatric fright convulsion”; and in Kaibao Bencao it is recorded to treat “all wind and dormant papules, stroke and hemiplegia, wry eye and mouth, angophrasia, tetany” etc.

With the development of modern medicine, people have realized that the most principal medicinal component of scorpion is derived from venom of scorpion, especially protein components in it, wherein the content of neurotoxins is highest. Action sites of these neurotoxins are various ion channels, including potassium ion channel, sodium ion channel, chlorine ion channel and calcium ion channel, etc.

Ion channels are direct participant in many life activities, such as muscular contraction of myocardium, skeletal muscle and smooth muscle etc, preventing T-cell activation, activating β-cell to release insulin, and transmitter transmission of many nerve cell synapses, etc. Therefore, the ion channels are closely linked with a variety of diseases, including cardiovascular diseases, epilepsy, neuropathy, tumor, rheumatoid arthritis and diabetes mellitus, etc. Scorpion toxin selectively acts on ion channels to alter their conductivity and kinetic characteristics etc and thereby exerting biological functions, having important application value in therapy of above-described diseases.

Based on the above characteristics, the scorpion toxin can be used as a molecular probe for study of the ion channels on cell membrane, it is helpful to having a deep understanding of regulation mechanism of the ion channels, and greatly promotes study on structure and function of the ion channels as well as related diseases; meanwhile provides important material for research fields such as physiology, pharmacology and neurobiology etc.

Scorpion toxin is mainly a kind of low molecular peptide with bioactivity consisting of 20 to 98 amino acids, it has an important development and application value, but because of source shortage of the scorpion and very low extraction rate of natural scorpion toxin, it is unable to meet the needs of clinic and research. Development of molecular biology and genetic engineering technology has made recombinant expression of scorpion toxin in other systems possible. Escherichia coli expression system, because of its advantages such as fast growth, high expression level, low cost, and simple operation, has become the most widely used expression system currently. But, because most of the scorpion toxins are rich in disulfide bond and the expression level in Escherichia coli is very low, it is difficult to be folded to a correct conformation, and often forms inclusion bodies, resulting in much lower bioactivity of the recombinant protein obtained than natural scorpion toxin. So, many measures are used in increasing soluble expression of the scorpion toxin. For example, use of secretory signal peptide, fusion expression technology and selection of some high copy expression vector and use of strong promoter, etc. In recent years, development of SUMO fusion technology greatly improved soluble expression of the scorpion toxin, but because of factors such as low yield of such method and low activity of the toxin obtained, it is inconvenient to perform further study on scorpion toxin and new drug development.

In order to increase yield and improve toxin activity, making the toxin structure refolded by oxidative renaturation in vitro is a good choice. In general, renaturating the inclusion body protein to an active protein requires the following three steps of treatment: first, harvesting and washing the inclusion body; second, dissolving the inclusion body, and obtaining a denaturated protein; third, renaturation. The denaturated proteins obtained through washing and dissolving have no bioactivity, they require renaturation. The principle of renaturation is restoring a target protein from fully extended denaturated state into a correct folded structure by slowly removing the denaturant and removing the reductant effect, making the disulfide bond correctly pairing to form a correct conformation, thereby producing a bioactivity. The yield of the first two steps is relatively high, and the yield of the third step is relatively low. There are many key factors that lead to a successful renaturation, including determining initial protein concentration of renaturation, renaturation buffer, pH, temperature, and renaturation interval, etc.

### SUMMARY OF THE INVENTION

The technical problem to be solved by the present invention is providing a method for efficiently isolating purified inclusion bodies of the recombinant scorpion venom protein, as well as a renaturation method for inclusion bodies of the scorpion venom protein.

Another technical problem to be solved by the present invention is providing the use of the scorpion venom protein obtained by the above-described method in preparation of anti-tumor drug.

In order to solve the above-described technical problem, the present invention adopts the following technical solution:

A method for purifying and renaturating scorpion venom protein, this method includes the following steps:

(1) putting inclusion bodies of scorpion venom protein into a denaturating buffer, stirring, centrifugating, filtering, collecting filtrate, to obtain a denaturated scorpion venom protein; for the stirring, its stirring speed is 50˜300 rpm, and stirring time is 1˜10 hours;

For the denaturating buffer, its composition is as follows: 50˜200 mmol/L Tris, 1˜10 mol/L Gdn-HCl, 20˜100 mmol/L DTT, pH 7˜9, the solvent is water; the composition of the denaturating buffer is preferably: 80˜120 mmol/L Tris, 4˜8 mol/L Gdn-HCl, 40˜60 mmol/L DTT, pH 7.5˜8.5; most preferably is: 100 mmol/L Tris, 6 mol/L Gdn-HCl, 50 mmol/L DTT, pH 8.0.

Wherein, Tris is a buffering agent for keeping pH value of the buffer; Gdn-HCl is a denaturant, it is able to destroy the hydrogen bond between the protein of the inclusion bodies by interaction between ions thereby solubilizing the protein; DTT is a reductant, it has antioxidant activity and can protect reducing group on the protein.

(2) purifying the denaturated scorpion venom protein;

(3) dissolving the denaturated scorpion venom protein with a renaturation buffer, performing a renaturation treatment, obtaining a renaturated scorpion venom protein;

for the renaturation buffer, its composition is as follows: 50˜200 mmol/L Na₂HPO₄, 10˜100 mmol/L Tris, 0.1˜1 mol/L L-Arg, 1˜5 mmol/L EDTA, 0.1˜5 mmol/L GSH, 0.05˜0.5 mmol/L GSSG, 5˜20% v/v glycerol, 0.01˜5% v/v tritonX-100, pH 7˜9, the solvent is water; the composition of the renaturation buffer preferably is: 80˜120 mmol/L Na₂HPO₄, 40˜60 mmol/L Tris, pH 7.5˜8.5, 0.4˜0.6 mol/L L-Arg, 1˜3 mmol/L EDTA, 0.5˜1.5 mmol/L GSH, 0.05˜0.2 mmol/L GSSG, 5˜10% v/v glycerol, 0.1˜0.3% v/v tritonX-100; most preferably is: 100 mmol/L Na₂HPO₄, 50 mmol/L Tris, pH 8.5, 0.5 mol/L L-Arg, 2 mmol/L EDTA, 1 mmol/L GSH, 0.1 mmol/L GSSG, 5% v/v glycerol, 0.2% v/v tritonX-100.

Wherein, Na₂HPO₄ and Tris are buffering agents for keeping pH value of the buffer; L-Arg is a cosolvent; EDTA is a metal ion chelating agent; GSH and GSSG can provide redox couple, and provide condition for formation of disulfide bond; tritonX-100 is a mild detergent, it can wash and remove membrane fragment and membrane protein; and glycerol is a protein stabilizer.

In step (1), the inclusion bodies of the scorpion venom protein are obtained by fermentation of recombinant Escherichia coli.

Wherein, the recombinant Escherichia coli is obtained by transforming Escherichia coli with expression plasmid having a cloned scorpion venom protein gene.

Wherein, the Escherichia coli is E. coli BL21 (DE3).

Wherein, the expression plasmid is pET29a.

Wherein, the expression plasmid having a cloned scorpion venom protein gene has His-tag at 5′-end or 3′-end of the scorpion venom protein gene, that is, the C-terminal or N-terminal of the scorpion venom protein obtained by expression have His-tag.

Preferably, the expression plasmid with cloned scorpion venom protein gene has His-tag at 5′-end of the scorpion venom protein gene, that is, the C-terminal of the scorpion venom protein obtained by expression has His-tag.

The His-tag in the present invention refers to a label consisting of multiple histidines, preferably six histidines.

A label (His-Tag) consisting of six histidines is respectively added at 5′-end or 3′-end of the scorpion venom protein gene, and the scorpion venom protein obtained by expression obtain are respectively designated as C_{His6}-rAGAP, N_{His6}-rAGAP, there is a substantial difference between the inhibitory activity of the C_{His6}-rAGAP and N_{His6}-rAGAP on growth of tumor cells, wherein, the inhibition rate of C_{His6}-rAGAP against HepG2 cells is up to 90%, whereas N_{His6}-rAGAP has almost no anti-tumor activity in vitro. Molecular dynamics simulation indicate that the His-tag of N-terminal floats at surface of fusion protein, altering surface charge of rAGAP, making surface electrostatic potential to change, whereas His-tag of C-terminal has small influence on three-dimensional structure and surface charge of AGAP.

Step (2) is purifying the denaturated scorpion venom protein obtained by a histidine affinity chromatography column.

In step (3), the renaturation treatment is incubating a renaturation buffer in which the denaturated scorpion venom protein being resolved at 4˜25□ for 12˜72 hours, preferably incubating at 15˜25□ for 18˜48 hours, most preferably incubating at 20□ for 24 hours.

The scorpion venom protein obtained by the above-described method for purifying and renaturating scorpion venom protein is also within protection scope of the present invention.

The use of above-described scorpion venom protein in preparation of anti-tumor drug is also within the protection scope of the present invention.

Beneficial effects: the present invention provides a method for efficiently isolating and purifying the recombinant scorpion venom protein inclusion bodies, and a method for renaturating the inclusion bodies of the scorpion venom protein. The present invention solved the problems such as low yield of the recombinant scorpion toxin in the Escherichia coli expression system, the protein being unable to correctly to be folded and low bioactivity, the bioactivity of the recombinant scorpion toxin obtained by present method is higher than the scorpion toxin expressed by the SUMO fusion technology in mice in vivo. By adopting the method of the present invention, calculated on basis of 1 L bacterial culture, finally about 16 mg of soluble scorpion toxin can be obtained, this is much higher than the protein obtained by other Escherichia coli expression system. Preparing the recombinant scorpion toxin protein by using the present invention method may increase the yield, save the cost, and easy to operate, providing convenience for further study of scorpion toxin and new drug development.

The anti-tumor effect of the recombinant scorpion toxin AGAP obtained by this inclusion body renaturation technology is higher than the recombinant AGAP obtained by the low temperature induction soluble expression technology and SUMO fusion expression technology reported in the foregoing literatures, and approached the activity level of natural scorpion toxin.

### BRIEF DESCRIPTION OF THE DRAWINGS

FIG. 1 is a diagram of constructed pET29a-AGAP recombinant plasmid.

FIG. 2 is result analysis of expression, purification and renaturation of C_{His6}-rAGAP. Figure A is a SDS-PAGE diagram of C_{His6}-rAGAP expression; lane 1: whole bacterial protein without IPTG induction; lane 2: whole bacterial protein induced by 1 mmol/L of IPTG at 37□ for 4 hours; lane 3: ultrasonic supernatant protein; lane 4, 5: ultrasonic precipitated protein, lane M: protein marker; Figure B is SDS-PAGE diagram of purification and renaturation of C_{His6}-rAGAP; lane 1: denaturated C_{His6}-rAGAP protein obtained by nickel column adsorption chromatography purification (diluted 20 fold with a renaturation buffer); lane 2: soluble C_{His6}-rAGAP protein after renaturation, concentration and centrifugation, lane M: protein marker; Figure C is purification of the renaturated C_{His6}-rAGAP protein by RP-HPLC (C4 column) analysis.

FIG. 3 is a diagram of comparing the inhibitory effect on growth of HepG2 tumor cells of C_{His6}-rAGAP and N_{His6}-rAGAP obtained by the present invention method obtain with rAGAP expressed by SUMO fusion technology.

FIG. 4 is inhibitory effect of different doses of C_{His6}-rAGAP on the tumor size in HepG2 mice model and influence on mice bodyweight. A is a photograph of the tumor taken out of nude mice; B is change of tumor volume in the nude mice determined every three days during drug administration, using 5-FU as a positive control; C is change of nude mice bodyweight determined every three days during drug administration. The statistical difference is analyzed by t-test, * indicates that there is a significant difference between rAGAP or 5-FU drug administration group with model group (p<0.05), ** indicates that there is a significant differencer between AGAP or 5-FU administration group with the model group (p<0.01).

FIG. 5 is inhibitory effect of C_{His6}-rAGAP (A) and N_{His6}-rAGAP (B) on voltage-sensitive sodium current in DRG neuron.

FIG. 6 is molecular simulations of AGAP, C_{His6}-rAGAP and N_{His6}-rAGAP. A is RMSD change of C_{α} atoms of C_{His6}-rAGAP and N_{His6}-rAGAP in molecular dynamics simulation (reference model is initial model); B, C is three-dimensional structure of C_{His6}-rAGAP and N_{His6}-rAGAP after the dynamic simulation; D, E are three-dimensional electrostatic potential surfaces of AGAP, C_{His6}-rAGAP and N_{His6}-rAGAP.

### DESCRIPTION OF THE EMBODIMENTS

Based on the following examples, the present invention can be better understood. However, one skilled in the art will understand that, the content described in the examples is only used to illustrate the invention, and it should not and will not restrict the invention defined in the claims.

### Example 1

### Construction of Recombinant Plasmid and Strain

(1) Construction of pET29a/C_{His6}-rAGAP Plasmid:

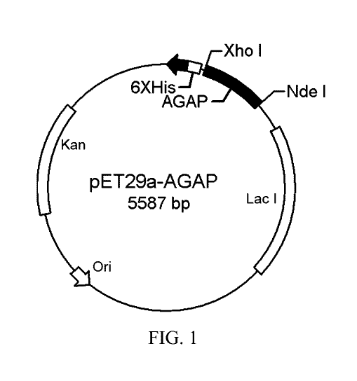

We entrusted GL Biochem (Shanghai) Ltd to synthesize mature peptide sequence of AGAP (this peptide sequence is well known by one skilled in the art), the target gene was amplificated by using P1, P2 primers. The primers had Nde I and Xho I restriction endonuclease recognition site, the target gene was clone into the pET29a plasmid, to obtain pET29a/C_{His6}-rAGAP recombinant plasmid (FIG. 1). The recombinant plasmid was transferred into E. coli DH5a, after screening in a LB culture medium containing 50 μg/ml kanamycin, a DNA sequencing was conducted to determine whether a target gene was correctly introduced into the strain or not. An amplification culture of the strain containing the recombinant plasmid were performed, the recombinant plasmid was extracted, and transferred into a E. coli BL21 (DE3) expression strain.

P1
(5′-GGAATTCCATATGGTACGCGATGGTTATATTGC-3′)
P2
(5′-CCGCTCGAGACCGCCATTGCATTTTCCTG-3′)

(2) Construction of pET43.1/N_{His6}-rAGAP Plasmid:

The scorpion toxin target gene was amplificated by using P3, P4 primer, the primers had Nde I and BamH I restriction endonuclease recognition site (marked by an italic underline), P3 primer had His-tag (marked by bold print), and His-tag was introduced into N-terminal of the AGAP, to obtain N_{His6}-AGAP gene. N_{His6}-AGAP gene was cloned into pET43.1 plasmid, to obtain pET43.1/N_{His6}-rAGAP recombinant plasmid. The recombinant plasmid was transferred into E. coli DH5a, after screening in a LB culture medium containing 50 μg/ml kanamycin a DNA sequencing was performed, to determine the target gene was whether correctly introduced into the strain. Amplification culture of the strain having the recombinant plasmid was performed, the recombinant plasmid was extracted and transferred into the E. coli BL21 (DE3) expression strain.

P3
(5′-GGAATTCCATATGCATCATCATCATCATCACGTACGCGATGGTT
AT ATTGC-3′)
P4
(5′-CGCGGATCCTTAACCGCCATTGCATTTTCCTG-3′)

### Example 2

### Expression and Purification of rAGAP

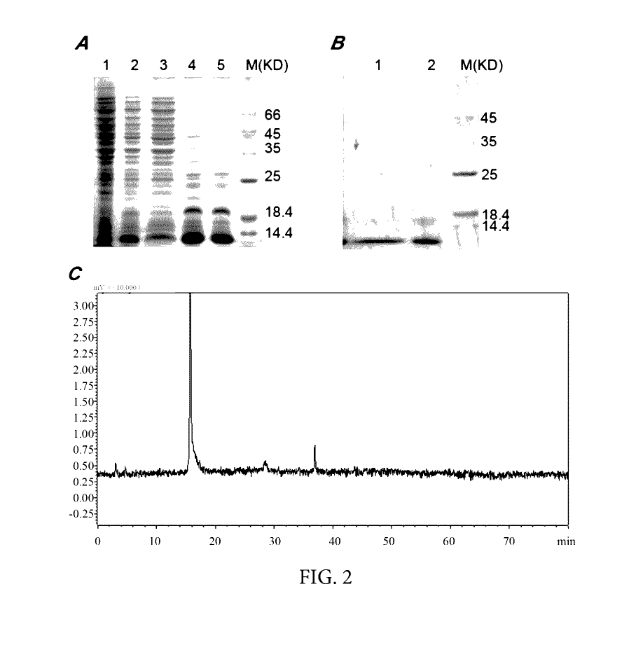

The E. coli BL21 (DE3) strains having the above recombinant plasmid were respectively cloned into a 50 mL of LB culture medium (containing 50 mg/mL kanamycin), and cultured at 37□ at 220 rpm overnight, the next day 25 mL of medium cultured overnight was removed to a 1 L of fresh LB culture medium and cultured to logarithmic growth phase. When absorbance OD₆₀₀ reached 0.6˜0.7, 1 mmol/L IPTG was added at 37□ and the expression of the target protein was induced for 4 hours. The bacterial liquid was centrifuged, and re-suspended with 100 mL lysis buffer (100 mmol/L NaCl, 50 mmol/L Tris, 2 mmol/L EDTA, 1% v/v tritonX-100, pH 8.0) , and sonicated in an ice bath at 400 w for 30 minutes. The cell lysis buffer was centrifugated at 12000 rpm for 10 minutes. SDS-PAGE founded that almost 90% of rAGAP was expressed in the form of inclusion body (FIG. 2A). The insoluble matter was re-suspended on a 50 mL eluent (100 mmol/L NaCl, 50 mmol/L Tris, 2 mmol/L EDTA, 2 mol/L Urea, pH 8.0), then sonicated for 5 minutes. After the centrifugation, the precipitate was washed with a deionized water for twice, then re-suspended with 10 mL buffer (100 mmol/L Tris, 6 mol/L Gdn-HCl, 50 mmol/L DTT, pH 8.0), mildly stirred for 4 hours, centrifugated at 12000 rpm for 10 minutes, then filtered with a 0.45 μm filtration membrane, the insoluble matter was removed, and DTT was removed from the supernatant with a Hi-Prep™ desalination column, the desalination column was first equilibrate with a buffer A (50 mmol/L Tris, 500 mmol/L NaCl, 8 mol/L urea, pH 8.0). The protein was eluted then applied to the histidine affinity chromatography column equilibrated with the buffer A, and gradient elution were performed successively by using 40, 60, 80, 100, 150, 300 and 500 mmol/L of imidazole eluents (in the eluent, 50 mmol/L Tris, 500 mmol/L NaCl, 8 mol/L urea, pH 8.0 remained unchanged). NaCl and imidazole was eliminated form the eluted rAGAP was with a Hi-Prep™ desalination column, the desalinating buffer was 50 mmol/L Tris, 8 mol/L urea, pH 8.0. After denaturation and purification, 75 mg of soluble scorpion toxin was obtained in 1 L of culture medium.

### Example 3

### Renaturation of rAGAP

The denaturated rAGAP obtained in Example 2 was dissolved in a renaturation buffer (100 mmol/L Na₂HPO₄, 50 mmol/L Tris, pH 8.5, 0.5 mol/L L-Arg, 2 mmol/L EDTA, 1 mmol/L GSH, 0.1 mmol/L GSSG, 5% v/v glycerol, 0.2% v/v triton-100) to a protein final concentration of 0.1 mg/ml, then incubated at 20□ for 24 hours. After centrifugation and filtration, the supernatant was concentrated 20 folds by Labscale TFF ultrafiltration system, dialyzed with 1×PBS, pH 7.4 buffer for 36 hours, the buffer was changed every 12 hours. After renaturation, 16 mg soluble scorpion toxin can be obtained in 1 L culture medium. The purification of rAGAP was identified by reverse-phase HPLC analysis, the purification may be up to 95% (FIG. 2B, C). The yield and purification in each step of C_{His6}-rAGAP purification can be seen in Table 1. The key factor in this example was concentration ratio of L-Arg concentration and GSH-GSSG. The method for purifying and renaturating N_{His6}-rAGAP was same as C_{His6}-rAGAP.

TABLE 1
Yield and purity in each step of C_{His6}-rAGAP purification

| | total weight^{#} | | |
|---|---|---|---|
| renaturation step | (mg) | purity* (%) | yield (%) |
| total cell extraction | 4520 | 35% | |
| washed inclusion | 3360 | 55% | 100.00% |
| bodies | | | |
| denaturated protein | 594 | 75% | 17.68% |
| nickle column | 305 | 85% | 9.10% |
| affinity | | | |
| chromatography | | | |
| renaturated protein | 77 | 85% | 2.30% |
| dialysis | 65 | 92% | 2.21% |

^{#}the weight of AGAP purified from 4 liters of culture medium for Escherichia coli (total wet weight of the cells was 13.41 g)
*purity of AGAP was analyzed by Quantity One (Bio-Rad) software

The key reagents in the renaturation buffer were GSH and GSSG reagents providing redox couple, its concentration optimization process was as follows:

The concentration of GSH was kept unchanged at 1 mmol/L,

1) adjusting pH to 9.0 and final concentration of GSSG to 0.05 mmol/L, and being renaturated at constant temperature of 20□ for 24 hours;

2) adjusting pH to 9.0 and final concentration of GSSG to 0.1 mmol/L, and being renaturated at constant temperature of 20□ for 24 hours;

3) adjusting pH to 9.0 and final concentration of GSSG to 0.2 mmol/L, and being renaturatied at constant temperature of 20□ for 24 hours;

4) adjusting pH to 9.0 and final concentration of GSSG to 0.3 mmol/L, being renaturated at constant temperature of 20□ for 24 hours;

5) adjusting pH to 9.0 and final concentration of GSSG to 0.4 mmol/L, being renaturated at constant temperature of 20□ for 24 hours;

6) adjusting pH to 8.5 and final concentration of GSSG to 0.05 mmol/L, being renaturated at constant temperature of 20□ for 24 hours;

7) adjusting pH to 8.5 and final concentration of GSSG to 0.1 mmol/L, being renaturated at constant temperature of 20□ for 24 hours;

8) adjusting pH to 8.5 and final concentration of GSSG to 0.2 mmol/L, being renaturated at constant temperature of 20□ for 24 hours;

9) adjusting pH to 8.5 and final concentration of GSSG to 0.3 mmol/L, being renaturated at constant temperature of 20□ for 24 hours;

10) adjusting pH to 8.5 and final concentration of GSSG to 0.4 mmol/L, being renaturated at constant temperature of 20□ for 24 hours;

11) adjusting pH to 8.0 and final concentration of GSSG to 0.05 mmol/L, being renaturated at constant temperature of 20□ for 24 hours;

12) adjusting pH to 8.0 and final concentration of GSSG to 0.1 mmol/L, being renaturated at constant temperature of 20□ for 24 hours;

13) adjusting pH to 8.0 and final concentration of GSSG to 0.2 mmol/L, being renaturated at constant temperature of 20□ for 24 hours;

14) adjusting pH to 8.0 and final concentration of GSSG to 0.3 mmol/L, being renaturated at constant temperature of 20□ for 24 hours;

15) adjusting pH to 8.0 and final concentration of GSSG to 0.4 mmol/L, being renaturated at constant temperature of 20□ for 24 hours;

16) adjusting pH to 7.5 and final concentration of GSSG to 0.05 mmol/L, being renaturated at constant temperature of 20□ for 24 hours;

17) adjusting pH to 7.5 and final concentration of GSSG to 0.1 mmol/L, being renaturated at constant temperature of 20□ for 24 hours;

18) adjusting pH to 7.5and final concentration of GSSG to 0.2 mmol/L, being renaturated at constant temperature of 20□ for 24 hours;

19) adjusting pH to 7.5 and final concentration of GSSG to 0.3 mmol/L, being renaturated at constant temperature of 20□ for 24 hours s;

20) adjusting pH to 7.5 and final concentration of GSSG to 0.4 mmol/L, being renaturated at constant temperature of 20° C. for 24 hours;

21) adjusting pH to 7.0 and final concentration of GSSG to 0.05 mmol/L, being renaturated at constant temperature of 20□ for 24 hours;

22) adjusting pH to 7.0 and final concentration of the GSSG to 0.1 mmol/L, being renaturated at constant temperature of 20□ for 24 hours;

23) adjusting pH to 7.0 and final concentration of GSSG was 0.2 mmol/L, a being renaturated at constant temperature of 20□ for 24 hours;

24) adjusting pH to 7.0 and final concentration of GSSG to 0 3 mmol/L, being renaturated at constant temperature of 20□ for 24 hours;

25) adjusting pH to 7.0 and final concentration of GSSG to 0 4 mmol/L, being renaturated at constant temperature of 20□ for 24 hours;

Base on the above pH and GSSG concentrations, it was found that when pH was 7.5˜8.5 and GSSG concentration was 0.05˜0.2 mmol/L, the renaturation efficiency of rAGAP was higher; wherein, when pH was 8.5 and concentration of GSSG was 0.1 mmol/L, the renaturation efficiency of the rAGAP was highest.

### Example 4

### Inhibitory Effect of rAGAP on Growth of HepG2 Cells

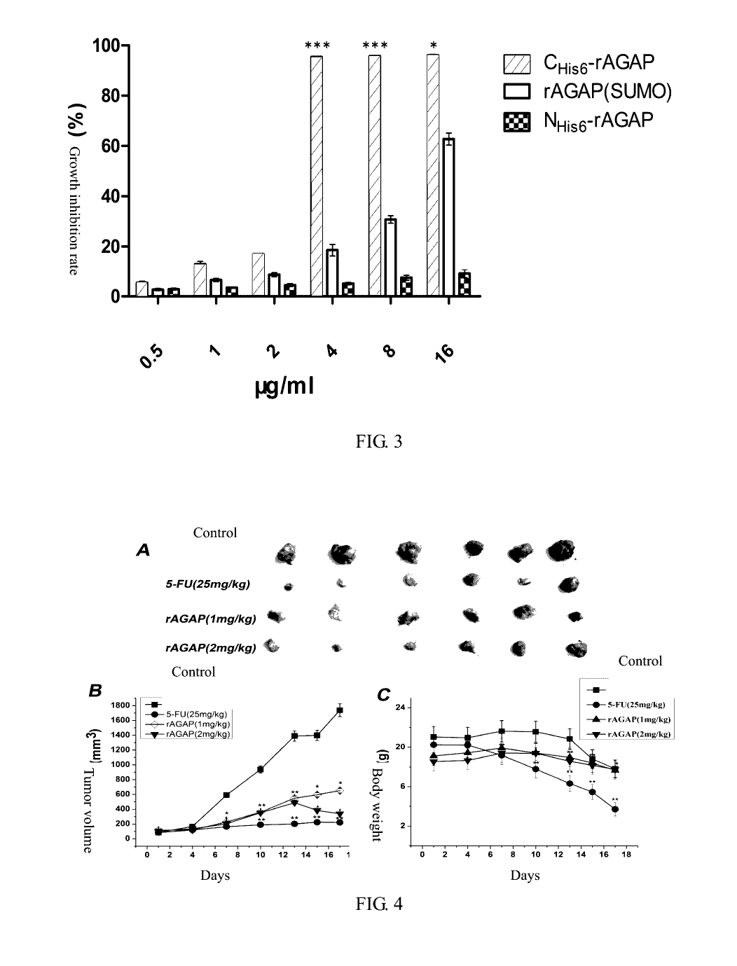

The HepG2 in logarithmic growth phase was transferred into a 96-well plate (2×10⁴ cells in each well), and cultured for 24 hours, then added into a serum-free medium and cultured for 12 hours, the cells were washed with a 1×PBS, pH 7.4 buffer, then rAGAP of different concentrations were added and incubated for 8 hours. The inhibitory effect of rAGAP on growth of HepG2 cells was determined by MTT method: to each well 10 μl MTT (5 mg/ml) was added, and incubated for 4 hours, the medium was removed, and 100 μl DMSO was added into each well, shaken at room temperature for 15 minutes, the absorbance was detected by a microplate reader at 570 nm, and inhibition rate of C_{His6}-rAGAP, N_{His6}-rAGAP and SUMO-rAGAP against HepG2 cells was calculated (FIG. 3). When concentration of rAGAP was 4 μg/ml, inhibition rate of C_{His6}-rAGAP obtained by present method against HepG2 cells reached 90%, whereas inhibition rate of the SUMO-rAGAP was only 20%, the N_{His6}-rAGAP almost had no anti-tumor activity in vitro. By calculation, IC₅₀ of the C_{His6}-rAGAP against HepG2 cells was 2.5 μg/ml, being six fold higher than the SUMO-rAGAP (15 μg/ml).

### Example 5

### Anti-Tumor Activity In Vivo of C_{His6}-rAGAP

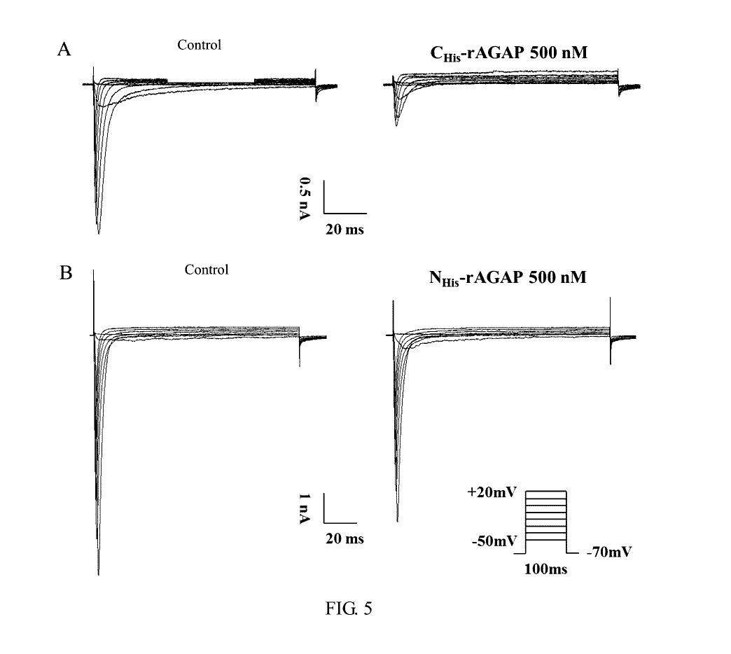

The CD-1 nu/nu mice (thymus removed, bodyweight 16˜18 g, half male and half female) were purchased from Shanghai Experiment Animal Center of Chinese Academy of Sciences. The mice were subcutaneously injected 5×10⁶ HepG2 cells to make a model, when the tumor volume was grew to about 100 mm³, the mice were randomized into four groups, 10 mice in each group, the experiment groups were respectively injected 1 mg/kg and 2 mg/kg of C_{His6}-rAGAP, the positive control group were injected 25 mg/kg of 5-FU, the negative control group was injected with normal saline, the volumes were all 200 μl/2 days. The change of tumor volume and bodyweight of the mice were determined every 2 days, after 18 days the mice were killed, and the tumor were taken out. The results demonstrated that, C_{His6}˜rAGAP of 2 mg/kg/2 d dose had an obvious inhibitory effect on mice tumor, when the dose was 1 mg/kg/2 d, the inhibitory effect was reduced (FIG. 4A, B). In order to evaluate the influence of C_{His6}-rAGAP and 5-FU on life quality of the mice, change of mice bodyweight was also determined during the experiment. The results demonstrated that, two groups of different dose C_{His6}-rAGAP had almost no influence on bodyweight of the mice, whereas bodyweight of the mice in 5-FU group were reduced significantly (FIG. 4C). Furthermore, after 18 days of drug administration, there was no death case in the mice of C_{His6}-rAGAP administration group, whereas 40% of the mice in 5-FU group were dead.

### Example 6

### Electrocortical Activity of C_{His6}-rAGAP.

An adult male C57 mice was killed by CO₂, lumbar (L4, 5 and 6) DRG neuron was taken out, and successively treated with trypsin□and collagenase□at 37□ for 30 minutes. The cells were mildly sucked and added to a DMEM medium supplemented 10% FBS for culture. The change of the membrane current was recorded with Axon 700A patch-clamp amplifier. In order to selectively record TTX-R sodium current, the composition of external liquid used was 35 mmol/L NaCl, 85 mmol/L choline-Cl, 20 mmol/L TEA-Cl, 3 mmol/L KCl, 1 mmol/L CaCl₂, 1 mmol/L MgCl₂, 10 mmol/L HEPES, 10 mmol/L D-glucose, and the pH was adjusted to 7.4 with Tris. The composition of the internal liquid used was 140 mmol/L CsF, 10 mmol/L NaCl, 10 mmol/L HEPES, 1 mmol/L EGTA, the pH was adjusted to 7.3 with CsOH. The DRG neuron was co-incubated with 500 nmol/L C_{His6}-rAGAP in a external liquid containing 300 nmol/L TTX for 2 minutes, the recorded TTX-R sodium current was as shown in FIG. 5, C_{His6}-rAGAP could inhibit about 70% of sodium current.

### Example 7

### Molecular Simulation of Three-Dimensional Structure of rAGAP

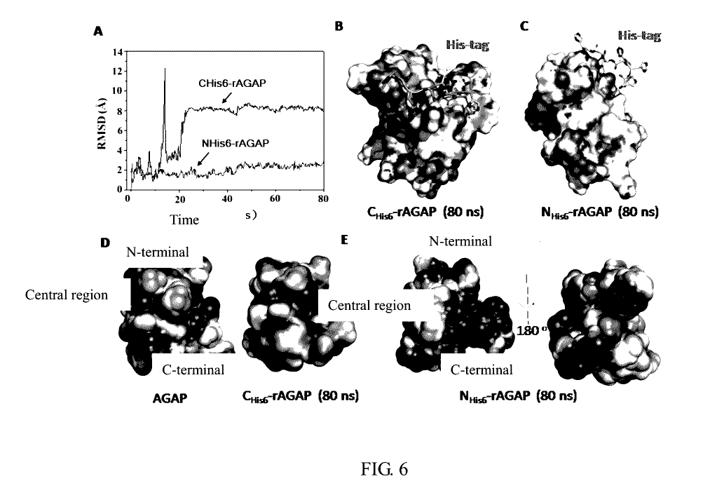

Using crystal structure of Lqh-alpha-IT (PDB ID: 2ATB) as a template, and AGAP was constructed by using Modeller software, then the molecular dynamics simulation to the three-dimensional structures of C_{His6}-rAGAP and N_{His6}-rAGAP were performed for 80 ns in Desmond by using OPLS-AA force field, to investigate the reason of C_{His6}-rAGAP activity being reserved whereas anti-tumor activity of N_{His6}-rAGAP being lost. RMSD analysis indicated that in the course of 80 ns of molecular dynamics simulation, His-tag at C-terminal underwent a large structural rearrangement, being close to AGAP surface, whereas the conformational change of His-tag at N-terminal was small, it floated at surface of AGAP (FIG. 6, A, B, C). Protein surface electrostatic potential analysis indicated that, compared with AGAP, influence of His-tag at C-terminal on surface electrostatic potential of C_{His6}-rAGAP was small, whereas influence of His-tag at N-terminal had a obvious influence on protein surface electrostatic potential (FIG. 6, D, E). Therefore, we infer that loss of N_{His6}-rAGAP activity may be closely related to change of the charge distribution at AGAP surface, and change of the charge distribution result in change of N_{His6}-rAGAP surface electrostatic potential.

1. A method for purifying and renaturating scorpion venom protein, the method comprising:
(1) putting inclusion bodies of scorpion venom protein into a denaturating buffer, stirring, centrifugating, and collecting a precipitate to obtain a denaturated scorpion venom protein; wherein the denaturating buffer has a pH of 7-9 and comprises 50-200 mmol/L Tris, 1-10 mol/L Gdn-HCl, 20-100 mmol/L DTT, and a solvent that is water;
(2) purifying the denaturated scorpion venom protein;
(3) dissolving the purified denaturated scorpion venom protein in a renaturation buffer, and renaturating the dissolved and purified denaturated scorpion venom protein to obtain a renaturated scorpion venom protein, wherein the renaturation buffer has a pH of 7-9 and comprises 50-200 mmol/L Na₂HPO₄, 10-100 mmol/L Tris, 0.1-1 mol/L L-Arg, 1-5 mmol/L EDTA, 0.1-5 mmol/L GSH, 0.05-0.5 mmol/L GSSG, 5-20% v/v glycerol, 0.01-5% v/v triton X-100, and a solvent that is water.

2. The method for purifying and renaturating scorpion venom protein according to claim 1, wherein the inclusion bodies of the scorpion venom protein are obtained by fermentation of recombinant Escherichia coli.

3. The method for purifying and renaturating scorpion venom protein according to claim 2, wherein the recombinant Escherichia coli is obtained by transforming Escherichia coli by an expression plasmid with cloned scorpion venom protein gene.

4. The method for purifying and renaturating scorpion venom protein according to claim 3, wherein the Escherichia coli is E. coli BL21 (DE3).

5. The method for purifying and renaturating scorpion venom protein according to claim 3, wherein the expression plasmid is pET29a.

6. The method for purifying and renaturating scorpion venom protein according to claim 3, wherein the expression plasmid with cloned scorpion venom protein gene has His-tag at 5′-end or 3′-end of the scorpion venom protein gene.

7. The method for purifying and renaturating scorpion venom protein according to claim 6, wherein the expression plasmid with cloned scorpion venom protein gene has His-tag at 5′-end of the scorpion venom protein gene.

8. The method for purifying and renaturating scorpion venom protein according to claim 1, wherein the denaturated scorpion venom protein is purified with a histidine affinity chromatography column.

9. The method for purifying and renaturating scorpion venom protein according to claim 1, wherein renaturating includes incubating the renaturation buffer and the dissolved and purified denaturated scorpion venom protein at 4-25° C. for 12-72 hours.

10. A scorpion venom protein obtained by the method for purifying and renaturating the scorpion venom protein according to claim 1.

11. An anti-tumor drug preparation, including the scorpion venom protein according to claim 10.
